(19) United States
(12) Patent Application Publication
(10) Pub. No.: US 2025/0367434 A1
(43) Pub. Date: Dec. 4, 2025

(54) ARRANGEMENT FOR ATTACHING AN EXTERNAL STIMULATOR TO A PATIENT AND METHODS OF USING

(71) Applicant: Boston Scientific Neuromodulation Corporation, Valencia, CA (US)

(72) Inventor: Thomas Gaviao Kilmar, Marina del Rey, CA (US)

(21) Appl. No.: 19/224,219

(22) Filed: May 30, 2025

Related U.S. Application Data
(60) Provisional application No. 63/655,927, filed on Jun. 4, 2024.

(51) Int. Cl. A61N 1/04; A61N 1/05; A61N 1/36

(52) U.S. Cl. CPC A61N 1/0496; A61N 1/05; A61N 1/36014; A61N 1/3605

(57) ABSTRACT

An external patch for a stimulation system includes a patch substrate configured for attachment to skin of a patient, the patch substrate having an interior side and an exterior side; at least one lead connector disposed on the interior side of the patch substrate and including connector contacts, each of the at least one lead connector configured to receive a proximal end portion of a stimulation lead for electrically coupling terminals of the stimulation lead to the connector contacts; patch contacts extending from the interior side of the patch substrate to the exterior side of the patch substrate and configured for electrically coupling to a stimulator; and conductors extending along, within, or on the interior side of the patch substrate and electrically coupling the patch contacts to the connector contacts.

### CROSS-REFERENCE TO RELATED APPLICATIONS

This application claims the benefit under 35 U.S.C. § 119(e) of U.S. Provisional Patent Application Ser. No. 63/655,927, filed Jun. 4, 2024, which is incorporated herein by reference.

### FIELD

The present invention is directed to the area of stimulation systems having an external stimulator attached to leads that are partially implanted and methods of making and using the systems. The present invention is also directed to an arrangement for coupling the external stimulator to the leads and attachment to the patient, as well as methods of making and using the arrangement.

### BACKGROUND

Implantable stimulation systems can provide therapeutic benefits in a variety of diseases and disorders. For example, electrical stimulation can be applied to the spinal cord using an implanted stimulation lead to provide, for example, spinal cord stimulation, to treat pain.

Stimulators have been developed to provide therapy for a variety of treatments. A stimulator can include a control module for generating electrical signals, one or more leads, and one or more electrodes on each lead. The lead is positioned near the nerves, muscles, or other tissue to be stimulated. The leads can be coupled to an implantable or external stimulator. In at least some instances, an external trial stimulator is used first to facilitate programming of stimulation and then an implantable stimulator is implanted and coupled to leads to provide long term therapy.

### BRIEF SUMMARY

One aspect is an external patch for a stimulation system that includes a patch substrate configured for attachment to skin of a patient, the patch substrate having an interior side and an exterior side; at least one lead connector disposed on the interior side of the patch substrate and including a plurality of connector contacts, each of the at least one lead connector configured to receive a proximal end portion of a stimulation lead for electrically coupling terminals of the stimulation lead to the connector contacts; a plurality of patch contacts extending from the interior side of the patch substrate to the exterior side of the patch substrate and configured for electrically coupling to a stimulator; and conductors extending along, within, or on the interior side of the patch substrate and electrically coupling the patch contacts to the connector contacts.

In at least some aspects, the external patch further includes at least one external electrode disposed on the interior side of the patch substrate. In at least some aspects, the at least one external electrode includes a return electrode for stimulation. In at least some aspects, the at least one external electrode includes a sensing electrode. In at least some aspects, the at least one external electrode further includes a return electrode for stimulation.

In at least some aspects, the external patch further includes a stimulator attachment interface disposed on the exterior side of the patch substrate and configured for attachment of an external stimulator thereto. In at least some aspects, the patch contacts include pogo pin contacts.

In at least some aspects, the external patch further includes adhesive disposed on at least the patch substrate for adhering the external patch to the skin of the patient. In at least some aspects, the external patch is water-resistant to resist flow of water through the external patch to the skin of the patient below the external patch. In at least some aspects, the external patch is configured to maintain a sterile field when attached to the skin of the patient in a sterile field.

Another aspect is a stimulation system that includes any of the external patches described above and an external stimulator electrically coupleable to the patch contacts of the external patch.

In at least some aspects, the external patch includes a stimulator attachment interface disposed on the exterior side of the patch substrate and configured for attachment of the external stimulator thereto. In at least some aspects, the stimulation system further includes at least one stimulation lead, wherein each of the at least one stimulation lead including a proximal end portion, a distal end portion, a plurality of electrodes disposed along the distal end portion, and a plurality of terminal disposed along the proximal end portion and electrically coupled to the electrodes. In at least some aspects, the external stimulator includes two base units; a flexible coupling section attached between the two base units and coupling the two base units to each other; a power source disposed in a one of the base units; and stimulation circuitry electrically coupled to the power source and disposed in at least one of the base units, wherein the stimulation circuitry is configured for generation of electrical stimulation signals to be delivered through one or more electrodes of at least one stimulation lead, when the at least one stimulation lead is electrically coupled to the external stimulator.

A further aspect is a stimulation system that includes any of the external patches described above and at least one stimulation lead, wherein each of the at least one stimulation lead including a proximal end portion, a distal end portion, a plurality of electrodes disposed along the distal end portion, and a plurality of terminal disposed along the proximal end portion and electrically coupled to the electrodes.

Yet another aspect is an external stimulator for a stimulation system that includes two base units; a flexible coupling section attached between the two base units and coupling the two base units to each other; a power source disposed in a one of the base units; and stimulation circuitry electrically coupled to the power source and disposed in at least one of the base units, wherein the stimulation circuitry is configured for generation of electrical stimulation signals to be delivered through one or more electrodes of at least one stimulation lead, when the at least one stimulation lead is electrically coupled to the external stimulator.

In at least some aspects, the power source is disposed in a first one of the two base units and the stimulation circuitry is disposed in a second one of the two base units.

Another aspect is a method for stimulating a patient. The method includes implanting a distal end portion of each of at least one stimulation lead in a patient, wherein electrodes are disposed along the distal end portion; attaching a proximal end portion of each of the at least one stimulation lead to any of the external patches described above, wherein terminals, which are electrically coupled to the electrodes, are disposed along the proximal end portion, wherein the attaching includes electrically coupling the terminals to the connector contacts of the external patch; adhering the external patch to skin of the patient; electrically coupling an external stimulator to the external patch; and delivering stimulation, generated by the external stimulator, through at least one of the electrodes of the at least one stimulation lead.

In at least some aspects, electrically coupling the external stimulator to the external patch includes attaching the external stimulator to the external patch. In at least some aspects, adhering the external patch includes forming a water-resistant seal using the external patch.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of a stimulation lead;

FIG. 3 is a schematic overview of one embodiment of components of an electrical stimulation system;

FIG. 4A is a schematic view towards an interior surface of one embodiment of an external patch;

FIG. 4B is a schematic view towards an exterior surface of the external patch of FIG. 4A coupled to proximal ends of two stimulation leads;

FIG. 4C is a schematic view towards an exterior surface of the external patch of FIG. 4A coupled to proximal ends of two stimulation leads with an external stimulator attached to the external patch; and

FIG. 5 is a schematic cross-sectional view of a portion of the external patch of FIG. 4A.

### DETAILED DESCRIPTION

The present invention is directed to the area of stimulation systems having an external stimulator attached to leads that are partially implanted and methods of making and using the systems. The present invention is also directed to an arrangement for coupling the external stimulator to the leads and attachment to the patient, as well as methods of making and using the arrangement.

Electrical stimulation systems and devices are used herein to exemplify the inventions, but it will be understood that these inventions can be utilized with other stimulation or modulation systems and devices. Examples of electrical stimulation systems include, but are not limited to, a least one lead with one or more electrodes disposed along a distal end portion of the lead and one or more terminals disposed along the one or more proximal end portions of the lead. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties.

Electrical stimulation systems are used in the description below but it will be understood that the electrical stimulation system, electrical stimulation lead, and electrodes can be replaced, in whole or in part, by an optical stimulation system (or electrical/optical stimulation system), optical stimulation lead, and optical emitters to produce optical stimulation or photobiomodulation. Examples of optical stimulation systems and electrical/optical stimulation systems, which include one or more optical emitters instead, or in addition to electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

An electrical, optical, or electrical/optical stimulation system can be used for a variety of applications including, but not limited to, spinal cord stimulation, deep brain stimulation, peripheral nerve stimulation (e.g., vagus nerve stimulation), other neural stimulation, muscle stimulation, and the like. Spinal cord stimulation will be used in the description below as an example, but it will be understood that any of the embodiments described below can be applied to other types of stimulation.

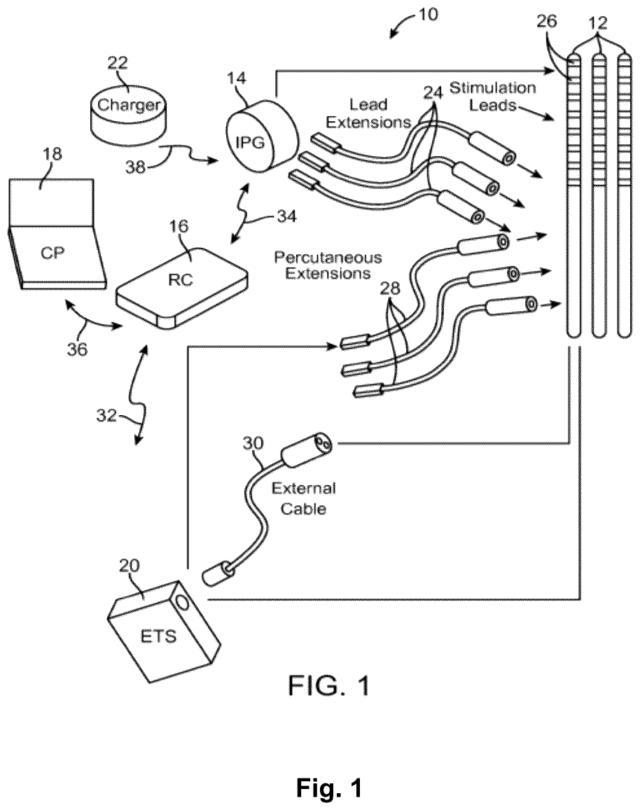

Turning to FIG. 1, one embodiment of an electrical stimulation system 10 includes one or more stimulation leads 12 and an implantable pulse generator (IPG) 14. The stimulation system 10 can also include one or more of an external remote control (RC) 16, a clinician's programmer (CP) 18, an external trial stimulator (ETS) 20, or an external charger 22. The IPG and ETS are examples of control modules for the electrical stimulation system. The ETS 20 is a type of external stimulator and will be used herein as an example, but it will be understood that any other external stimulator can be used in the place of the ETS 20.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the array of electrodes 26 in accordance with a set of stimulation parameter values. The IPG 14 can be implanted into a patient's body, for example, below the patient's clavicle area or within the patient's abdominal cavity or at any other suitable site. The IPG 14 or ETS 20 can have multiple stimulation channels which may be independently programmable to control the magnitude of the current stimulus from each channel. In some embodiments, the IPG 14 can have any suitable number of stimulation channels including, but not limited to, 4, 6, 8, 12, 16, 32, or more stimulation channels. The IPG 14 can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the array of electrodes 26 in accordance with a set of stimulation parameter values. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 is also applicable to the ETS 20.

The RC 16 may be used to telemetrically communicate with or control the IPG 14 or ETS 20 via a uni- or bi-directional wireless communications link 32 or via any other wired or wireless communication technique. Once the IPG 14 and neurostimulation leads 12 are implanted, the RC 16 may be used to telemetrically communicate with or control the IPG 14 via a uni- or bi-directional communications link 34 or via any other wired or wireless communication technique. Such communication or control allows the IPG 14, for example, to be turned on or off and to be programmed with different stimulation parameter sets. The IPG 14 or ETS 20 may be operated to modify the programmed stimulation parameter values to actively control the characteristics of the electrical stimulation energy output by the IPG 14 or ETS 20. In at least some embodiments, the CP 18 (or RC 16 or other programming device) allows a user, such as a clinician, the ability to program stimulation parameter values for the IPG 14 and ETS 20 in the operating room and in follow-up sessions. Alternately, or additionally, in at least some embodiments, stimulation parameter values can be programed via wireless communications (e.g., Bluetooth) between the RC 16 (or other external device such as a hand-held electronic device like a mobile phone, tablet, or the like) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). In at least some embodiments, the stimulation parameter values provided by the CP 18 are also used to program the RC 16, so that the stimulation parameter values can be subsequently modified by operation of the RC 16 in a stand-alone mode (i.e., without the assistance of the CP 18). The CP 18 or RC 16 can be any suitable device including, but not limited to, a computer or other computing device, laptop, mobile device (for example, a mobile phone or tablet), or the like or any combination thereof. The CP 18 or RC 16 can include software applications for interacting with the IPG 14 or ETS 20 and for programming the IPG 14 or ETS 20.

Additional examples of the RC 16, CP 18, ETS 20, and external charger 22 can be found in the references cited herein as well as U.S. Pat. Nos. 6,895,280; 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036 as well as any of the other references cited herein, all of which are incorporated herein by reference in their entireties.

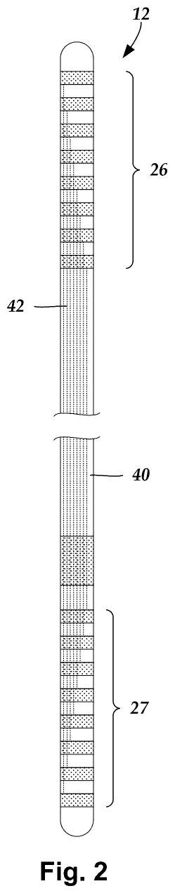

FIG. 2 shows, in schematic side view, one embodiment of a lead 12 suitable for implanting into a patient and providing electrical stimulation. In some embodiments, the lead 12 is coupled directly to a control module. In other embodiments, the lead 12 is coupled to the IPG 14 or ETS 20 via one or more lead extensions 24, 28. In the illustrated embodiment of FIG. 2, an array of electrodes 26 is disposed along a distal portion of a lead body 40 of the lead 12 and an array of lead terminals 27 is disposed along a proximal portion of the lead body. Lead conductors, such as lead conductor 42, extend along a longitudinal length of the lead and electrically couple the array of electrodes 26 to the array lead terminals 27. Conductors can extend along the longitudinal length of the lead within one or more lumens defined in the lead. In other instances, the conductors may extend along the lead within the lead body itself. Additional examples of stimulation leads can be found in the references cited herein.

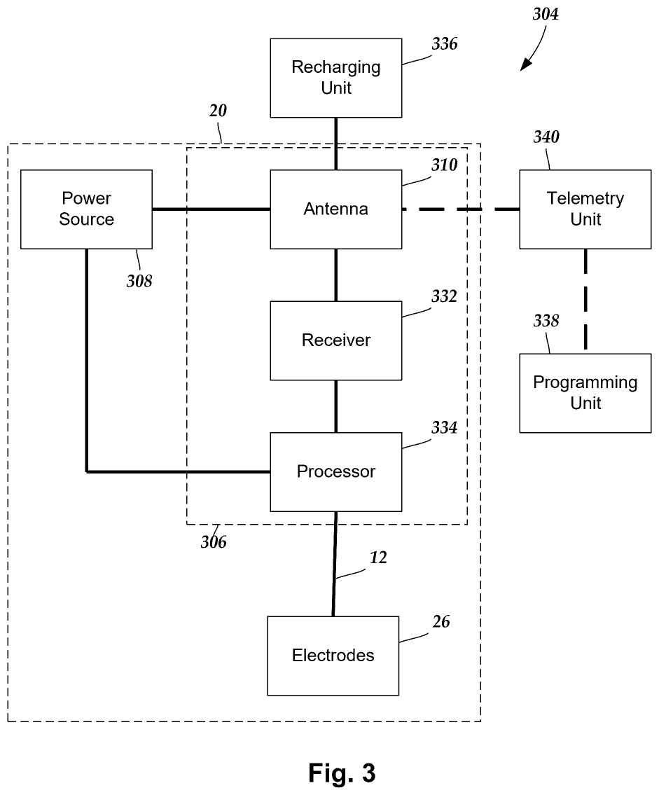

FIG. 3 is a schematic overview of one embodiment of components of an electrical stimulation arrangement 304 that includes one or more leads 12 and an ETS 20 with stimulation circuitry 306 (which may include an antenna 310) and a power source 308. It will be understood that the electrical stimulation arrangement can include more, fewer, or different components and can have a variety of different configurations including those configurations disclosed in the stimulator references cited herein.

If the power source 308 is a rechargeable battery or chargeable capacitor, the power source may be recharged/charged using the antenna 310, if desired. Power can be provided for recharging/charging by inductively coupling the power source 308 through the antenna 310 to a recharging unit 336 external to the user. Examples of such arrangements can be found in the references identified above.

In at least some embodiments, electrical current is emitted by the electrodes 26 on the lead 12 to stimulate nerve fibers, muscle fibers, or other body tissues near the electrodes. In this way, in these embodiments, electrical current is applied to the patient via the electrodes. The stimulation circuitry 306 can include, among other components, a processor 334 and an optional receiver 332. The processor 334 is included to control the timing and electrical characteristics of the ETS. For example, the processor 334 can, if desired, control one or more of the timing, frequency, strength, duration, and waveform of the pulses. In addition, the processor 334 can select which electrodes can be used to provide stimulation, if desired (see, for example, U.S. Pat. No. 8,412,345, which is incorporated herein by reference in its entirety). In at least some embodiments, the processor 334 selects which electrode(s) are cathodes and which electrode(s) are anodes. In at least some embodiments, the processor 334 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 338 that, for example, allows modification of pulse characteristics. In the illustrated embodiment, the processor 334 is coupled to a receiver 332 which, in turn, is coupled to the antenna 310. This allows the processor 334 to receive instructions from an external source to, for example, direct the pulse characteristics and the selection of electrodes, if desired.

In at least some embodiments, the antenna 310 is capable of receiving signals (e.g., RF signals) from an external telemetry unit 340 that is programmed by the programming unit 338. The programming unit 338 can be external to, or part of, the telemetry unit 340. The telemetry unit 340 can be a device that can be carried by the user and can have a form similar to a pager, cellular phone, or remote control, if desired. As another alternative, the telemetry unit 340 may not be worn or carried by the user but may only be available at a home station or at a clinician's office. The programming unit 338 can be any unit that can provide information to the telemetry unit 340 for transmission to the ETS 20. The programming unit 338 can be part of the telemetry unit 340 or can provide signals or information to the telemetry unit 340 via a wireless or wired connection. One example of a suitable programming unit is a computer operated by the user or clinician to send signals to the telemetry unit 340.

The signals sent to the processor 334 via the antenna 310 and the receiver 332 can be used to modify or otherwise direct the operation of the ETS 20. For example, the signals may be used to modify the pulses of the ETS 20 such as modifying one or more of pulse duration, pulse frequency, pulse waveform, and pulse strength. The signals may also direct the ETS 20 to cease operation, to start operation, to start charging the battery, or to stop charging the battery.

Optionally, the ETS 20 may include a transmitter (not shown) coupled to the processor 334 and the antenna 310 for transmitting signals back to the telemetry unit 340 or another unit capable of receiving the signals. For example, the ETS 20 may transmit signals indicating whether the ETS 20 is operating properly or not or indicating when the battery needs to be charged or the level of charge remaining in the battery. The processor 334 may also be capable of transmitting information about the pulse characteristics so that a user or clinician can determine or verify the characteristics.

An ETS 20 is often used during an initial programming and testing period to test efficacy of the therapy or to determine stimulation parameters that will be used later with an IPG 14 (or at least to facilitate later programming of the IPG). In at least some cases, the initial programming and testing period using the ETS 20 is at least 6, 8, 12, 18, or 20 hours or 1, 2, 5, 7, 10, 14, 15, 21, 28, 30, 31, or more days. In these instances, the distal portion of each lead 12, where the electrodes 26 reside, is implanted at the stimulation site. The proximal portion of each lead 12, where the terminals 27 reside, extend out of an incision through the skin of the patient and are attached (or attachable) to the ETS 20.

Often, the proximal portion of the lead(s) 12 and the ETS 20 are attached to the skin of the patient. As an example, for spinal cord stimulation, the proximal portion of the lead(s) 12 and the ETS 20 can be taped to the patient's back or torso. This arrangement can be difficult or awkward for the patient, particularly when the initial programming and testing period extends for days. For example, the patient may be directed not to shower or bathe to avoid damage to the lead(s) 12 or ETS 20 or to avoid infection. In at least some instances, the ETS 20 has a form factor that can be uncomfortable to the patient when attached to the patient's body, for example, when sleeping. Moreover, the proximal end portion of the lead 12 often does not remain sterile so that each lead 12 must be replaced with a new lead when the IPG 14 is implanted.

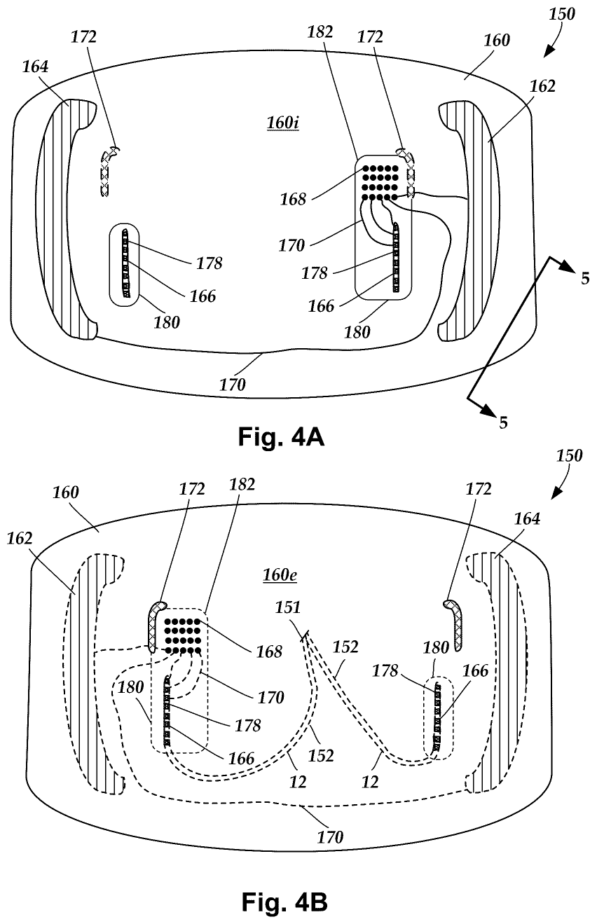

As described herein, an external patch 150 can be used to attach the ETS 20 and proximal end portion 152 of the lead(s) 12 to the skin of the patient and electrically couple the leads to the ETS, as illustrated in FIGS. 4A to 4C. In at least some embodiments, the external patch 150 can provide a waterproof or water-resistant barrier. In at least some embodiments, the external patch 150 can maintain a sterile field. In at least some embodiments, the external patch 150 is disposable or single-use and the ETS 20 is reusable. In addition, in at least some embodiments, the form factor of the ETS 20 is modified for comfort of the patient.

FIG. 4A illustrates a view from the interior side of an external patch 150, which may be used, for example, for spinal cord stimulation (or any other suitable type of stimulation) for placement on the back of the patient. FIG. 4B illustrates a view from the exterior side of the external patch 150, attached to a patient, with two leads 12 extending from an incision 151 and coupled to the external patch. FIG. 4C illustrates a view from the exterior side of the external patch 150 with an ETS 20 attached to the external patch. In at least some embodiments, the ETS 20 has two base units 156 connected by a coupling section 158 (e.g., a tether), as described in more detail below. In at least some embodiments, the two base units 156 are attachable to the external patch 150 for positioning the two base units 156 on opposite sides of the spinal cord for patient comfort.

The external patch 150 includes a patch substrate 160 (e.g., a patch film), one or more optional external electrodes 162, 164, one or more lead connectors 166, patch contacts 168, conductors 170, and an optional stimulator attachment interface 172. The conductors electrically couple the lead connectors 166 and external electrode(s) 162, 164 to the patch contacts 168. The external patch 150 may be disposable and, at least in some embodiments, is replaceable by another external patch periodically during the use of the ETS 20.

The patch substrate 160 has an interior side 160i that is placed against the skin of the patient and an exterior side 160e to which, in at least some embodiments, the ETS 20 is attached. The patch substrate 160 can be a thin, flexible sheet that can be made of any suitable material, such as, for example, silicone rubber, thermoplastic polyurethane, thermoplastic elastomer, thermoplastic rubber, polyethylene or the like or any combination thereof. In at least some embodiments, the material is biocompatible and passes a skin sensitization test or the like. In at least some embodiments, the patch substrate 160 has a thickness of up to 3, 5, or 6 mm. The thickness may vary along the substrate. For example, in at least some embodiments, edges may be relatively thin and portions where other components are attached may be thicker. In at least some embodiments, the patch substrate 160 is a multi-layer substrate, which includes multiple films attached to each other. In at least some embodiments, the material and thickness of the patch substrate 160 are selected to resist or prevent tearing under expected usage conditions and still remain flexible to resist or prevent disengagement from the skin of the patient as the patient moves. In at least some embodiments, the material and thickness of the patch substrate 160 are selected to permit stretching of the patch substrate 160 under expected usage conditions.

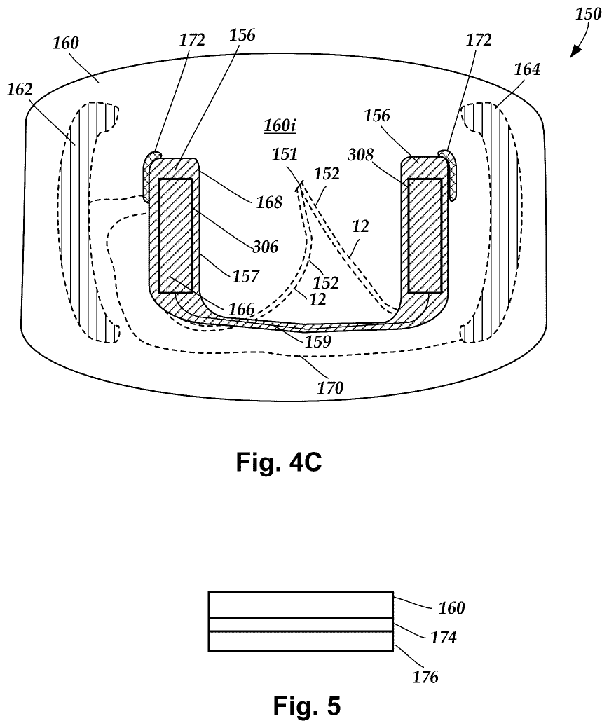

A portion of the patch substrate 160 has adhesive 174 disposed thereon, as illustrated by the cross-section of a portion of the external patch 150 in FIG. 5. Examples of suitable adhesive 174 include, but are not limited to, any suitable medical adhesive, such as, any biocompatible adhesive or texture that provides skin adhesion or the like. In at least some embodiments, the adhesive 174 is disposed around an entire perimeter of the patch substrate 160 or around one or more portions of the perimeter of the patch substrate. In at least some embodiments, the adhesive is disposed on at least 25%, 33%, 50%, 66%, 75%, 80%, or 90% or more of the surface area of the patch substrate 160 (or of the patch substrate 160 and other components of the external patch 150 that are to be disposed against the skin of the patient). Any other suitable disposition of adhesive 174 can be used. In at least some embodiments, prior to application of the patch substrate 160 to the patient, a release liner 176 is disposed over the adhesive 174, as illustrated in FIG. 5, to prevent or resist adhesion of the external patch 150 to other surfaces. In at least some embodiments, the adhesive 174 and patch substrate 160 are selected to resist or prevent loss of adhesion to the skin of the patient under expected usage conditions, including expected movements of the patient.

In at least some embodiments the patch substrate 160 is water-resistant or waterproof. In at least some embodiments, the adhesive 174 is water-resistant or waterproof. A water-resistant or waterproof seal can prevent infection or reduce the likelihood of infection. In at least some embodiments, the patch substrate 160 and adhesive 174 provide a seal when applied to the skin of the patient so that, at least in some instances or embodiments, a sterile field can be maintained beneath the external patch 150. In at least some embodiments, the proximal portions of the lead(s) 12 (and elements of the external patch 150 that are positioned on the side of the patch substrate 160 next to the patient) remain sterile during use of the external patch. In at least some embodiments, when the proximal portion 152 of the lead(s) 12 remain sterile, the lead(s) will not need to be replaced after the stimulation period with the ETS 20 so that the lead(s) can be fully implanted and used with the IPG 14.

One or more lead connectors 166 are attached to the interior surface 160i of the patch substrate 160. Each lead connector 166 includes connector contacts 178 that are individually arranged to make electrical contact with at least one of the terminals 27 (FIG. 2) disposed along the proximal end portion of a lead 12. Any suitable lead connector 166, which maintains the connector contacts 178 in electrical contact with the terminals 27 of the lead under expected usage conditions, can be used In at least some embodiments, the lead connector 166 can include a connector substrate 180, such as a flexible circuit board, silicone rubber substrate, or the like, with the connector contacts 178 disposed on the connector substrate. The lead connector 166 can include one or more fasteners to fasten the lead 12 to the connector substrate 180. Examples of suitable fasteners include, but are not limited to, a sleeve, a sheath, tape, a compliant spring that holds the lead in place (e.g., using friction, pressure, or geometry or any combination thereof), or the like or any combination thereof. In at least some embodiments, the lead connector 178 can be the same or similar to a lead connector of a lead extension (see, for example, the lead extensions described in the electrical, optical, or electrical/optical stimulation systems of any of the references cited above as well as U.S. Pat. Nos. 7,244,150; 8,224,450; 8,600,507; 8,682,439; 8,849,396; 8,897,876; 10,905,871; 10,918,873; and 11,139,603 and U.S. Patent Application Publications Nos. 2012/0053646; 2014/0148885; 2015/0209575; 2016/0059019; and 2023/0056675, all of which are incorporated herein by reference in their entireties).

In at least some embodiments, the external patch 150 includes one or more external electrodes 162, 164 attached to, or otherwise disposed on, the interior surface 160i of the patch substrate 160. Any suitable number of external electrodes 162, 164 can be used including, but not limited. In at least some embodiments, an external electrode 162, 164 is printed or otherwise formed on the patch substrate 160. In at least some embodiments, an external electrode 162, 164 is an unsupported metal piece attached to the patch substrate 160 by adhesive or the like. In at least some embodiments, an external electrode 162, 164 is printed or otherwise formed on a substrate (for example, a flexible circuit board or silicone rubber substrate,) and attached to the patch substrate 160 by adhesive or the like. An external electrode 162, 164 can have any suitable size or shape and, when multiple external electrodes are present, the external electrodes can be the same or different with respect to size, shape, or the like.

Each external electrode 162, 164 is arranged to make contact with the skin of the patient when the external patch 150 is attached to the patient. In at least some embodiments, an external electrode (for example, external electrode 162) can be used as a stimulation electrode that is remote from the electrodes 26 of the lead(s) 12. For example, the external electrode 162, 164 can be used, similar to the case of the IPG 14, as a remote electrode. In at least some embodiments, such as for monopolar stimulation, the external electrode 162, 164 can be a return electrode for stimulation signals delivered through one or more of the electrodes 26. At least some conventional ETS's do not have the capability of acting as a remote electrode, which can limit the ability to perform monopolar stimulation using the ETS.

In at least some embodiments, an external electrode (for example, external electrode 164) can be used as a sensing electrode, for example, as a sensing reference electrode. In at least some embodiments, the external patch 150 with an electoral electrode 164 can provide the ability to sense during the programming and testing period.

The external patch 150 includes patch contacts 168 that extend through the patch substrate 160 from the interior surface 160i to the exterior surface 160e. In at least some embodiments, the patch contacts 168 are disposed on a substrate 182 (which may be the same substrate as a connector substrate 180, as illustrated in FIG. 4A), such as a flexible circuit board or the like. Any suitable type of contact can be used for the patch contacts 168. Examples of patch contacts 168 include, but are not limited to, contact pads, biased contacts (for example, pogo pin contacts), a connector (for example, a USB or other type of connector), or the like or any combination thereof.

In at least some embodiments, the patch contacts 168 extend through openings in the patch substrate 160. In at least some embodiments, the patch substrate 160 is sealed (for example, using adhesive or the like) to the patch contacts 168, the substrate 182, or both to provide a waterproof or water-resistant seal.

The patch contacts 168 are electrically coupled to the connector contacts 178 of the lead connectors 166 and the external electrode(s) 162, 164 by conductors 170. For clarity of illustration, only a few of the conductors 170 are illustrated in FIGS. 4A, 4B, and 4C. The conductors 170 can be wires, conductive traces printed or otherwise disposed on the patch substrate 160 or other substrate (for example, a flexible circuit board or silicone rubber substrate, that is adhered to the patch substrate 160) or the like, or any other suitable conductors or any combination thereof (for example, a conductor 170 can be a combination of a wire and trace(s)). In at least some embodiments, the conductors 170 are embedded (or partially embedded) in the patch substrate 160. In at least some embodiments with a multi-layer patch substate 160, the conductors 170 are disposed between layers of the patch substrate.

In at least some embodiments, the external patch 150 includes a stimulator attachment interface 172 disposed on the exterior surface 160e of the patch substrate 160 for attaching (for example, releasably attaching) the ETS 20 to the external patch 150. The attachment of the ETS 20 includes electrical contact between the patch contacts of the external patch 150 and corresponding contacts of the ETS. The size, shape, and arrangement of the stimulator attachment interface 172 can depend on the form of the ETS 20. In the illustrated embodiment, the stimulator attachment interface 172 includes two couplers that are arranged to receive and retain respective portions of the ETS 20. In at least some embodiments, the stimulator attachment interface 172 is made using rigid or flexible plastic (or any combination thereof) and attached to the patch substrate using, for example, a permanent adhesive or any other attachment material or mechanism.

The stimulator attachment interface 172, optionally in conjunction with the ETS 20, can utilize any suitable retention mechanism including, but not limited to, clip(s), detent(s), snap fit arrangements, interference fit arrangements, adhesive, hook and loop fastener(s), or the like or any combination thereof. In at least some embodiments, the retention mechanism is arranged to maintain attachment of the ETS 20 to the external patch 150 during expected activity of the patient and allow the patient or other individual to remove the ETS without pulling the external patch 150 away from the skin of the patient. This can allow the patient to bathe or shower by temporarily detaching the ETS 20 from the external patch 150. In other embodiments, the stimulator attachment interface 172 can permanently attach the ETS 20 to the external patch 150. In at least some embodiments, instead of attaching the ETS 20 directly to the external patch 150, a connector with an attached cord extending from the ETS 20 is attached to the external patch 150.

An external patch 150, as described herein, can be made for any suitable ETS 20 including known and commercially available external stimulators. A new ETS 20, illustrated in FIG. 4C, is designed for use with an external patch 150 and is particularly adapted for spinal cord stimulation or other stimulation where the external patch is attached or attachable to the back of the patient. The ETS 20 of FIG. 4C includes a housing 157 defining two base units 156 connected by a flexible coupling section 158. This ETS 20 and the external patch 150 allows the placement of the external patch and ETS on the back of the patient with the base units 156, when attached to the external patch, disposed over portions of the back on opposite sides of the spine with the flexible coupling section 158 traversing the spine. In at least some embodiments, the base units 156 may be positioned over muscle when the patient lays on his back. In addition, in at least some embodiments, the two base units 156 can have a limited height, relative to the back of the patient, to provide a more comfortable fit than a conventional external stimulator attached to the back of the patient.

In at least some embodiments, the power source 308 (see also, FIG. 3) is disposed in one of the base units 156 and the stimulation circuitry 306 (see also, FIG. 3) is disposed in the other ETS unit, as illustrated in FIG. 4C. In some embodiments, the stimulation circuitry 306 is distributed between the two base units 156. For example, the power source 308 and antenna 310 (FIG. 3) are in one ETS unit and the remainder of the stimulation circuitry 306 is in the other ETS unit. In all of these embodiments, one or more conductors 159 (only one of which is illustrated in FIG. 4C for clarity of illustration), such as one or more wires or one or more traces disposed on a substrate (e.g., a flexible circuit board or silicone rubber substrate,), extend through the coupling section 158 and electrically couple the operative portions of the ETS 20 (e.g., the power source 308 and the stimulation circuitry 306). The housing 157 of the ETS 20 is arranged to couple to the stimulator attachment interface 172 according to the selected retention mechanism.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. An external patch for a stimulation system, the external patch comprising:
a patch substrate configured for attachment to skin of a patient, the patch substrate comprising an interior side and an exterior side;
at least one lead connector disposed on the interior side of the patch substrate and comprising a plurality of connector contacts, each of the at least one lead connector configured to receive a proximal end portion of a stimulation lead for electrically coupling terminals of the stimulation lead to the connector contacts;
a plurality of patch contacts extending from the interior side of the patch substrate to the exterior side of the patch substrate and configured for electrically coupling to a stimulator; and
conductors extending along, within, or on the interior side of the patch substrate and electrically coupling the patch contacts to the connector contacts.

2. The external patch of claim 1, further comprising at least one external electrode disposed on the interior side of the patch substrate.

3. The external patch of claim 2, wherein the at least one external electrode comprises a return electrode for stimulation.

4. The external patch of claim 2, wherein the at least one external electrode comprises a sensing electrode.

5. The external patch of claim 4, wherein the at least one external electrode further comprises a return electrode for stimulation.

6. The external patch of claim 1, further comprising a stimulator attachment interface disposed on the exterior side of the patch substrate and configured for attachment of an external stimulator thereto.

7. The external patch of claim 1, wherein the patch contacts comprise pogo pin contacts.

8. The external patch of claim 1, further comprising adhesive disposed on at least the patch substrate for adhering the external patch to the skin of the patient.

9. The external patch of claim 8, wherein the external patch is water-resistant to resist flow of water through the external patch to the skin of the patient below the external patch.

10. The external patch of claim 8, wherein the external patch is configured to maintain a sterile field when attached to the skin of the patient in a sterile field.

11. A stimulation system, comprising:
the external patch of claim 1; and
an external stimulator electrically coupleable to the patch contacts of the external patch.

12. The stimulation system of claim 11, wherein the external patch comprises a stimulator attachment interface disposed on the exterior side of the patch substrate and configured for attachment of the external stimulator thereto.

13. The stimulation system of claim 11, further comprising at least one stimulation lead, wherein each of the at least one stimulation lead comprising a proximal end portion, a distal end portion, a plurality of electrodes disposed along the distal end portion, and a plurality of terminal disposed along the proximal end portion and electrically coupled to the electrodes.

14. The stimulation system of claim 11, wherein the external stimulator comprises
two base units;
a flexible coupling section attached between the two base units and coupling the two base units to each other;
a power source disposed in a one of the base units; and
stimulation circuitry electrically coupled to the power source and disposed in at least one of the base units, wherein the stimulation circuitry is configured for generation of electrical stimulation signals to be delivered through one or more electrodes of at least one stimulation lead, when the at least one stimulation lead is electrically coupled to the external stimulator.

15. A stimulation system, comprising:
the external patch of claim 1; and
at least one stimulation lead, wherein each of the at least one stimulation lead comprises a proximal end portion, a distal end portion, a plurality of electrodes disposed along the distal end portion, and a plurality of terminal disposed along the proximal end portion and electrically coupled to the electrodes.

16. An external stimulator for a stimulation system, comprising:
two base units;
a flexible coupling section attached between the two base units and coupling the two base units to each other;
a power source disposed in a one of the base units; and
stimulation circuitry electrically coupled to the power source and disposed in at least one of the base units, wherein the stimulation circuitry is configured for generation of electrical stimulation signals to be delivered through one or more electrodes of at least one stimulation lead, when the at least one stimulation lead is electrically coupled to the external stimulator.

17. The external stimulator of claim 16, wherein the power source is disposed in a first one of the two base units and the stimulation circuitry is disposed in a second one of the two base units.

18. A method for stimulating a patient, the method comprising
implanting a distal end portion of each of at least one stimulation lead in a patient, wherein electrodes are disposed along the distal end portion;
attaching a proximal end portion of each of the at least one stimulation lead to the external patch of claim 1, wherein terminals, which are electrically coupled to the electrodes, are disposed along the proximal end portion, wherein the attaching comprises electrically coupling the terminals to the connector contacts of the external patch;
adhering the external patch to skin of the patient;
electrically coupling an external stimulator to the external patch; and
delivering stimulation, generated by the external stimulator, through at least one of the electrodes of the at least one stimulation lead.

19. The method of claim 18, wherein electrically coupling the external stimulator to the external patch comprises attaching the external stimulator to the external patch.

20. The method of claim 18, wherein adhering the external patch comprises forming a water-resistant seal using the external patch.
